(19) United States
(12) Patent Application Publication
(10) Pub. No.: US 2026/0235570 A1
(43) Pub. Date: Aug. 13, 2026

(54) SIMULTANEOUS DETECTION OF A PLURALITY OF COMPOUNDS USING DUAL IONIZATION SOURCE

(71) Applicant: SHIMADZU CORPORATION, Kyoto-shi (JP)

(72) Inventors: Nitish SURYAWANSHI, Mumbai (IN); Shalu NAIR, Mumbai (IN); Samruddha CHAVAN, Mumbai (IN); Nitin SHUKLA, Mumbai (IN); Jitendra KELKAR, Mumbai (IN); Pratap RASAM, Mumbai (IN)

(73) Assignee: SHIMADZU CORPORATION, Kyoto-shi (JP)

(21) Appl. No.: 19/531,958

(22) Filed: Feb. 6, 2026

(30) Foreign Application Priority Data
Feb. 10, 2025 (IN) 202511010856

(51) Int. Cl. G01N 33/15; H01J 49/16

(52) U.S. Cl. CPC G01N 33/15; H01J 49/165; H01J 49/168

(57) ABSTRACT

The present disclosure refers to a method of detecting a plurality of compounds in a sample which comprises first ionizing the sample with a first ionization method, and second ionizing the sample with a second ionization method.

### BACKGROUND

Electrospray ionization (ESI) is a widely used ionization technique due to its applicability to most of the polar to mid-polar molecules. On the other hand, atmospheric chemical ionization (APCI) is less often explored ionization technique. Due to the presence of polar functional groups on the polar molecules, it readily acquires charge whereas nonpolar molecules do not readily acquire charge and need higher voltage which is provided by corona discharge in APCI. APCI recently became popular in pharma for Nitrosamines testing using LC-MS/MS.

N-nitrosamines have been monitored extensively in formulation free from NDSRIs, considering the risk of pharmaceuticals since 2018. Since 2020, there has been an increased number of reports of more structurally complex N-nitrosamines related to the structure of the active substance itself in several drug products. These are often referred to as nitrosamine drug substance related impurities (NDSRIs) and may be formed by nitrosation of an amine moiety present in the active substance. After N-nitrosamines, the US Food and Drug Administration (FDA) now demands the pharmaceutical manufacturers to establish if their active pharmaceutical substance (API) or associated and regulatory implications, there is an increasing demand for testing of both these type of molecules in API and formulations.

Nitrosamine even though being very small mostly polar to mid polar molecules were widely analyzed by using APCI across the industry due to several reasons such as its low molecular weight, attracting high background or noise, poor selectivity due to high background at trace levels, which is a demand due to dynamic regulatory requirements etc. Whereas APCI comparatively showed better sensitivity, selectivity and accuracy and now is often used for nitrosamine testing. There are other classes of nitrosamine-like molecules which are also monitored by the industry called nitrosamine drug substance related impurities (NDSRIs). Unlike the general nitrosamines, these are mostly analyzed by using ESI. This is due to reasons such as relatively high molecular weight providing better selectivity, whereas many NDSRIs are having structural similarities to that of active pharmaceutical ingredients (API) facilitating easy ionization sites delivering higher order of sensitivity. The preferential use of ionization units for a separate testing of nitrosamines and NDSRIs significantly affects time, resources, chemical consumption, and overall throughput.

### SUMMARY

In one aspect, the present disclosure relates to a single method for simultaneous detection of both N-nitrosamines and NDSRI by using LC-MS/MS. In another aspect, the present disclosure relates to a novel LC-MS/MS ionization source enabling selective and sensitive detection of both N-nitrosamines and NDSRI. In another aspect, the present disclosure relates to a simple and sensitive LC-MS/MS method to quantify a plurality of N-nitrosamines impurities and NDSRI without any complex sample pre-treatment.

In another aspect, the present disclosure provides a method of detecting a plurality of compounds in a sample, the method comprising first ionizing the sample with a first ionization method to produce a first ionized compound, and second ionizing the sample with a second ionization method to produce a second ionized compound, and detecting the first and second ionized compounds. In some embodiments, the first and second ionized compounds are detected in one device. In some embodiments, the first and second ionized compounds are detected at the same location. In some embodiments, the first and second ionized compounds are detected simultaneously. In some embodiments, the first ionization method is Electrospray Ionization (ESI) method. In some embodiments, the second ionization method is Atmospheric Pressure Chemical Ionization (APCI) method. In some embodiments, the method further comprises performing liquid chromatography on the sample prior to the first and second ionizing. In some embodiments, the method further comprises performing mass spectrometry on the sample ionized with the first and second ionization methods. In some embodiments, the method further comprises performing tandem mass spectrometry on the sample ionized with the first and second ionization methods. In some embodiments, the first ionizing comprises applying a first voltage to at least one compound of the plurality of compounds that is readily ionizable under ESI method. In some embodiments, the second ionizing comprises applying a second voltage to at least one compound of the plurality of compounds that is readily ionizable under APCI conditions. In some embodiments, the method is performed in one device.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 shows a graphical representation of ESI and APCI voltage change programed.

FIG. 2 shows a schematic diagram of sample and spiked sample preparation.

FIG. 3(A) shows UV chromatogram of Afatinib API spiked sample.

FIG. 3(B) shows MS chromatogram of Nitrosamines and N-AFA sample.

FIG. 4(A) shows chromatograms for LOQ level solution for NDEA.

FIG. 4(B) shows calibration curve for NDEA.

FIG. 4(C) shows chromatograms for LOQ level solution for NDMA.

FIG. 4(D) shows calibration curve for NDMA

FIG. 4(E) shows chromatograms for LOQ level solution for N-AFA.

FIG. 4(F) shows calibration curve for N-AFA.

### DETAILED DESCRIPTION

Hereinafter, the present disclosure will be described in more detail to help the understanding of the present disclosure.

When the term “about” is used, it is used to mean a certain effect or result can be obtained within a certain tolerance, and the skilled person knows how to obtain the tolerance. When the term “about” is used in describing a value or an end-point of a range, the disclosure should be understood to include the specific value or end-point referred to. In one aspect, the term “about” means plus or minus 20, 19, 18, 17, 16, 15, 14, 13, 12, 11, 10, 9, 8, 7, 6, 5, 4, 3, 2, or 1% of the numerical value of the number with which it is being used.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed embodiment. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

Further, unless expressly stated to the contrary, “or” and “and/or” refers to an inclusive and not to an exclusive. For example, a condition A or B, or A and/or B, is satisfied by any one of the following: A is true (or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

As used herein, the term “chromatography” refers to a process in which a chemical mixture carried by a liquid or gas is separated into components as a result of differential distribution of the chemical entities as they flow around or over a stationary liquid or solid phase.

In one aspect, the present disclosure provides a method of detecting a compound in a sample. In some embodiments, the method detects a plurality of compounds in a sample. In some embodiments, the method comprises first ionizing the sample with a first ionization method to produce a first ionized compound, and second ionizing the sample with a second ionization method to produce a second ionized compound. In some embodiments, the method detects the first and second ionized compounds. In some embodiments, the method detects the first ionized compounds. In some embodiments, the method detects the second ionized compounds. In some embodiments, the present disclosure provides a method of detecting a plurality of compounds in a sample, the method comprises first ionizing the sample with a first ionization method to produce a first ionized compound, second ionizing the sample with a second ionization method to produce a second ionized compound, and detecting the first and second ionized compounds.

In some embodiments, detecting, discovering, determining, measuring, evaluating, counting, and assessing a compound are used interchangeably and may include quantitative and/or qualitative determinations, including, for example, identifying the compound, determining presence and/or absence of the compound, and quantifying the compound.

Herein the “sample” may include a quantity of material from a biological, environmental, medical, or patient source in which separation, purification, detection, measurement, or analysis is sought. In some embodiments, the sample may be food, drug, or cosmetic. A sample may be an intermediate sample obtained during a manufacturing process for the same. A sample may be a stored sample obtained after storing the same. On the other hand, it is meant to include both biological and environmental samples. A sample may include a specimen of synthetic origin. Environmental samples include environmental material, such as surface matter, soil, water and industrial samples, as well as samples obtained from food and dairy processing instruments, apparatus, equipment, utensils, disposable and non-disposable items. In some embodiments, the food sample may be selected from the group consisting of cured meats, processed fish, cocoa, beer, and other alcoholic beverages. In some embodiments, the cosmetic sample may be selected from the group consisting of lotions and shampoos. In some embodiments, the sample may a pesticide. In some embodiments, the sample may be an amine drug or a pharmaceutically acceptable salt hereof. In some embodiments, the sample may be blood pressure medicine.

In some embodiments, the sample comprises a nitrosamine. In some embodiments, the nitrosamine comprises an N-nitrosamine. In some embodiments, the N-nitrosamine comprises at least 1, 2, 3, 4, 5, 6, 7, 8 or 9 N-nitrosamine(s) selected from the group consisting of N-nitroso-dimethylamine (NDMA), N-nitrosomethylethylamine (NMEA), N-Nitrosopyrrolidine (NPYR), N-nitroso-diethylamine (NDEA), N-nitrosopiperidine (NPIP), N-nitroso-ethyl-isopropylamine (NEIPA), N-nitroso-diisopropylamine (NDIPA), N-nitroso-dipropylamine (NDPA), and N-nitroso-dibutylamine (NDBA). In some embodiments, the N-nitrosamine described herein consists essentially of N-nitroso-dimethylamine (NDMA), N-nitrosomethylethylamine (NMEA), N-Nitrosopyrrolidine (NPYR), N-nitroso-diethylamine (NDEA), N-nitrosopiperidine (NPIP), N-nitroso-ethyl-isopropylamine (NEIPA), N-nitroso-diisopropylamine (NDIPA), N-nitroso-dipropylamine (NDPA), and N-nitroso-dibutylamine (NDBA). In some embodiments, the nitrosamine has a molecular weight of about 500, 400, 300, 200, 100, 50 g/mol or lower, and/or of about 10, 20, 30, 40, 50, 60, 70, 80, 90, 100 g/mol or higher. In some embodiments, the sample contains about 10, 20, 30, 40, 50, 60, 70, 80, 90, 100, 150, 200 ppb or more and/or about 1000, 900, 800, 700, 600, 500, 400, 300, 200 ppb or less of each nitrosamine. In some embodiments, the sample contains about 1000, 2000, 3000 ppb or more and/or about 9000, 8000, 7000, 6000, 5000, 4000, 3000, 2000, 1000 ppb or less of total nitrosamine.

In some embodiments, the sample comprises a nitrosamine drug substance related impurity (NDSRI). The nitrosamine drug substance-related impurities (NDSRIs), for example in the composition of drug product, may be formed by nitrosating reaction between amines (primary, secondary, tertiary, or quaternary amines) and/or excipients. In some embodiments, the sample comprises at least 1, 2, 3, 4, 5, 6, 7, 8, 9, 10, 11, 12, 13, 14 or 15 different NDSRI(s). In some embodiments, the NDSRI comprises N-nitroso Afatinib impurity-2 (N-AFA). In some embodiments, the NDSRI has a molecular weight of about 400, 500, 600, 700, 800, 900, 1000 g/mol or higher, and/or of about 10000, 9000, 8000, 700, 6000, 5000, 4000, 3000, 2000, 1000 g/mol or lower. In some embodiments, the sample contains about 10, 20, 30, 40, 50, 60, 70, 80, 90, 100, 150, 200 ppb or more and/or about 1000, 900, 800, 700, 600, 500, 400, 300, 200 ppb or less of each NDSRI. In some embodiments, the sample contains about 1000, 2000, 3000 ppb or more and/or about 9000, 8000, 7000, 6000, 5000, 4000, 3000, 2000, 1000 ppb or less of total NDSRI.

In some embodiments, the first ionization method and the second ionization method are different. In some embodiments, the first ionizing is performed prior to the second ionizing. In some embodiments, the second ionizing is performed prior to the first ionizing. In some embodiments, the first and second ionizing steps are performed alternately. In some embodiments, the first and second ionizing steps are performed simultaneously. In some embodiments, the first and second ionizing steps are not performed simultaneously. In some embodiments, the first and second ionizing steps are switched. In some embodiments, the first and second ionizing steps are continuously switched.

In some embodiments, the first ionization method is Electrospray Ionization (ESI) method. The ESI method may create charged ions from a liquid sample. In the ESI method according to some embodiments, a liquid sample is introduced to a tip of a thin nozzle, and a high voltage is applied to the tip of the nozzle. As a result, a strong unequal electric field may be formed at the tip of the nozzle, and the liquid sample may be nebulized as charged droplets by the strong electric field. Further, the droplets may be divided by Coulomb force of ions in the droplets, thereby being ionized. ESI may be selected for analysis of high-polarity compounds which are typically found in drugs and pesticides.

In some embodiments, the second ionization method is Atmospheric Pressure Chemical Ionization (APCI) method. The APCI method may vaporize and ionize a sample at atmospheric pressure using corona discharge. For example, the APCI method may ionize solvent molecules that transfer charge to analytes in a sample. In the APCI method according to some embodiments, a gas flow in a nebulizer forces the liquid sample to be nebulized. Then, the nebulized liquid sample is heated to evaporate solvent in the droplets. Thereafter, buffer ions may be produced by the corona discharge to ionize the sample, resulting in chemical ionization. APCI may be selected for compounds having lower polarity.

In some embodiments, the method described herein further comprises performing liquid chromatography on the sample prior to the first and second ionizing. In some embodiments, the liquid chromatography comprises high-performance liquid chromatography (HPCL). In some embodiments, the liquid chromatography comprises fast protein liquid chromatography (FPCL). the liquid chromatography comprises liquid-liquid chromatography.

As used herein, the term “liquid chromatography” or “LC” refers a process of selective retardation of one or more components of a fluid solution as the fluid uniformly percolates through a column of a finely divided substance, or through capillary passageways. U.S. Patent Application Publication NO. 2003/0136904 describes an exemplary liquid chromatography and is incorporated herein by reference for its entirety.

The retardation may result from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography.

In some embodiments, the method further comprises performing mass spectrometry on the sample ionized with the first and second ionization methods. In some embodiments, the method further comprises performing tandem mass spectrometry on the sample ionized with the first and second ionization methods.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer, a mass analyzer, and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”).

In some embodiments, the first ionizing comprises selecting a first voltage to be applied to the sample. In some embodiments, the first voltage comprises a first interface voltage. In some embodiments, the first interface voltage is from 0.1 kV to 2 kV. Herein “interface voltage” may include the electrical potential applied at the interface between the liquid chromatography system and the mass spectrometer, including the voltage used to spray the liquid sample into the gas phase for ionization, for example, within an electrospray ionization (ESI). In some embodiments, the first interface voltage is from 0.1 kV to 2 kV. In some embodiments, the first interface voltage is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 kV. In some embodiments, the first interface voltage is at most about 50, 40, 30, 20, 15, 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3, 0.2, or 0.1 kV. In some embodiments, the first interface voltage ranges from 0.01 to 50, 0.1 to 50, 0.1 to 40, 0.1 to 30, 0.1 to 20, 0.1 to 10, 0.1 to 9.5, 0.1 to 9, 0.1 to 8.5, 0.1 to 8, 0.1 to 7.5, 0.1 to 7, 0.1 to 6.5, 0.1 to 6, 0.1 to 5.5, 0.1 to 5, 0.1 to 4.5, 0.1 to 4, 0.1 to 3.5, 0.1 to 3, 0.1 to 2.5, 0.1 to 2, 0.1 to 1.5, 0.1 to 1, 0.1 to 0.9, 0.1 to 0.8, 0.1 to 0.7, 0.1 to 0.6, 0.1 to 0.5, 0.1 to 0.4, 0.2 to 10, 0.2 to 9.5, 0.2 to 9, 0.2 to 8.5, 0.2 to 8, 0.2 to 7.5, 0.2 to 7, 0.2 to 6.5, 0.2 to 6, 0.2 to 5.5, 0.2 to 5, 0.2 to 4.5, 0.2 to 4, 0.2 to 3.5, 0.2 to 3, 0.2 to 2.5, 0.2 to 2, 0.2 to 1.5, 0.2 to 1, 0.2 to 0.9, 0.2 to 0.8, 0.2 to 0.7, 0.2 to 0.6, 0.2 to 0.5, 0.2 to 0.4, 0.3 to 10, 0.3 to 9.5, 0.3 to 9, 0.3 to 8.5, 0.3 to 8, 0.3 to 7.5, 0.3 to 7, 0.3 to 6.5, 0.3 to 6, 0.3 to 5.5, 0.3 to 5, 0.3 to 4.5, 0.3 to 4, 0.3 to 3.5, 0.3 to 3, 0.3 to 2.5, 0.3 to 2, 0.3 to 1.5, 0.3 to 1, 0.5 to 10, 0.5 to 9.5, 0.5 to 9, 0.5 to 8.5, 0.5 to 8, 0.5 to 7.5, 0.5 to 7, 0.5 to 6.5, 0.5 to 6, 0.5 to 5.5, 0.5 to 5, 0.5 to 4.5, 0.5 to 4, 0.5 to 3.5, 0.5 to 3, 0.5 to 2.5, 0.5 to 2, 0.5 to 1.5, 0.5 to 1, 0.5 to 0.9, 0.5 to 0.8, 0.5 to 0.7, or 0.5 to 0.6 kV. In some embodiments, the first voltage comprises a first focus voltage. In some embodiments, the first focus voltage is from 1 kV to 3 kV. In some embodiments, the first focus voltage is at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 kV. In some embodiments, the first focus voltage is at most about 50, 40, 30, 20, 15, 10, 9.5, 9, 8.5, 8, 7.5, 7, 6.5, 6, 5.5, 5, 4.5, 4, 3.5, 3, 2.5, 2, 1.5, or 1 kV. In some embodiments, the first focus voltage ranges from 0.1 to 30, 0.1 to 20, 0.1 to 10, 0.1 to 9.5, 0.1 to 9, 0.1 to 8.5, 0.1 to 8, 0.1 to 7.5, 0.1 to 7, 0.1 to 6.5, 0.1 to 6, 0.1 to 5.5, 0.1 to 5, 0.1 to 4.5, 0.1 to 4, 0.1 to 3.5, 0.1 to 3, 0.1 to 2.5, 0.1 to 2, 0.1 to 1.5, 0.1 to 1, 0.1 to 0.9, 0.1 to 0.8, 0.1 to 0.7, 0.1 to 0.6, 0.1 to 0.5, 0.1 to 0.4, 0.5 to 30, 0.5 to 20, 0.5 to 10, 0.5 to 9.5, 0.5 to 9, 0.5 to 8.5, 0.5 to 8, 0.5 to 7.5, 0.5 to 7, 0.5 to 6.5, 0.5 to 6, 0.5 to 5.5, 0.5 to 5, 0.5 to 4.5, 0.5 to 4, 0.5 to 3.5, 0.5 to 3, 0.5 to 2.5, 0.5 to 2, 0.5 to 1.5, 0.5 to 1, 0.5 to 0.9, 0.5 to 0.8, 0.5 to 0.7, 0.5 to 0.6, 0.7 to 30, 0.7 to 20, 0.7 to 10, 0.7 to 9.5, 0.7 to 9, 0.7 to 8.5, 0.7 to 8, 0.7 to 7.5, 0.7 to 7, 0.7 to 6.5, 0.7 to 6, 0.7 to 5.5, 0.7 to 5, 0.7 to 4.5, 0.7 to 4, 0.7 to 3.5, 0.7 to 3, 0.7 to 2.5, 0.7 to 2, 0.7 to 1.5, 0.7 to 1, 1 to 30, 1 to 20, 1 to 10, 1 to 9.5, 1 to 9, 1 to 8.5, 1 to 8, 1 to 7.5, 1 to 7, 1 to 6.5, 1 to 6, 1 to 5.5, 1 to 5, 1 to 4.5, 1 to 4, 1 to 3.5, 1 to 3, 1 to 2.5, 1 to 2, 1 to 1.5, 2 to 30, 2 to 20, 2 to 10, 2 to 9.5, 2 to 9, 2 to 8.5, 2 to 8, 2 to 7.5, 2 to 7, 2 to 6.5, 2 to 6, 2 to 5.5, 2 to 5, 2 to 4.5, 2 to 4, 2 to 3.5, 2 to 3, or 2 to 2.5 kV.

In some embodiments, the first ionizing described herein comprises applying a first voltage to the sample. The first voltage may have been predetermined prior to performing the method described herein. In some embodiments, the first ionizing comprises applying a first voltage to at least one compound of the plurality of compounds that is readily ionizable under ESI method. In some embodiments, the first ionizing comprises applying a first voltage to at least one NDSRI in the sample. In some embodiments, the first ionizing comprises applying a first voltage to all compounds in the sample that are readily ionizable by ESI method, irrespective of their elution. In some embodiments, the first ionizing comprises applying a first voltage to all NDSRI in the sample.

In some embodiments, the second ionizing comprises selecting a second voltage to be applied to the sample. The second voltage may have been predetermined prior to performing the method described herein. In some embodiments, the second voltage comprises a second interface voltage. In some embodiments, the second interface voltage is from 0.5 kV to 1.5 kV. In some embodiments, the second interface voltage is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 kV. In some embodiments, the second interface voltage is at most about 50, 40, 30, 20, 15, 10, 9.5, 9, 8.5, 8, 7.5, 7, 6.5, 6, 5.5, 5, 4.5, 4, 3.5, 3, 2.5, 2, 1.5, 1, 0.9, 0.8, 0.7, 0.6, 0.5, 0.4, 0.3, 0.2, or 0.1 kV. In some embodiments, the second interface voltage ranges from 0.01 to 50, 0.1 to 50, 0.1 to 40, 0.1 to 30, 0.1 to 20, 0.1 to 10, 0.1 to 9.5, 0.1 to 9, 0.1 to 8.5, 0.1 to 8, 0.1 to 7.5, 0.1 to 7, 0.1 to 6.5, 0.1 to 6, 0.1 to 5.5, 0.1 to 5, 0.1 to 4.5, 0.1 to 4, 0.1 to 3.5, 0.1 to 3, 0.1 to 2.5, 0.1 to 2, 0.1 to 1.5, 0.1 to 1, 0.1 to 0.9, 0.1 to 0.8, 0.1 to 0.7, 0.1 to 0.6, 0.1 to 0.5, 0.1 to 0.4, 0.2 to 10, 0.2 to 9.5, 0.2 to 9, 0.2 to 8.5, 0.2 to 8, 0.2 to 7.5, 0.2 to 7, 0.2 to 6.5, 0.2 to 6, 0.2 to 5.5, 0.2 to 5, 0.2 to 4.5, 0.2 to 4, 0.2 to 3.5, 0.2 to 3, 0.2 to 2.5, 0.2 to 2, 0.2 to 1.5, 0.2 to 1, 0.2 to 0.9, 0.2 to 0.8, 0.2 to 0.7, 0.2 to 0.6, 0.2 to 0.5, 0.2 to 0.4, 0.3 to 10, 0.3 to 9.5, 0.3 to 9, 0.3 to 8.5, 0.3 to 8, 0.3 to 7.5, 0.3 to 7, 0.3 to 6.5, 0.3 to 6, 0.3 to 5.5, 0.3 to 5, 0.3 to 4.5, 0.3 to 4, 0.3 to 3.5, 0.3 to 3, 0.3 to 2.5, 0.3 to 2, 0.3 to 1.5, 0.3 to 1, 0.5 to 10, 0.5 to 9.5, 0.5 to 9, 0.5 to 8.5, 0.5 to 8, 0.5 to 7.5, 0.5 to 7, 0.5 to 6.5, 0.5 to 6, 0.5 to 5.5, 0.5 to 5, 0.5 to 4.5, 0.5 to 4, 0.5 to 3.5, 0.5 to 3, 0.5 to 2.5, 0.5 to 2, 0.5 to 1.5, 0.5 to 1, 0.5 to 0.9, 0.5 to 0.8, 0.5 to 0.7, 0.5 to 0.6, 1 to 10, 1 to 9.5, 1 to 9, 1 to 8.5, 1 to 8, 1 to 7.5, 1 to 7, 1 to 6.5, 1 to 6, 1 to 5.5, 1 to 5, 1 to 4.5, 1 to 4, 1 to 3.5, 1 to 3, 1 to 2.5, 1 to 2, 1 to 1.5, 1 to 1.4, 1 to 1.3, 1 to 1.2, or 1 to 1.1 kV. In some embodiments, the second voltage comprises a second focus voltage. In some embodiments, the second focus voltage is from 3 kV to 5 kV. In some embodiments, the second focus voltage is at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 kV. In some embodiments, the second focus voltage is at most about 50, 40, 30, 20, 15, 10, 9.5, 9, 8.5, 8, 7.5, 7, 6.5, 6, 5.5, 5, 4.5, 4, 3.5, 3, 2.5, 2, 1.5, or 1 kV. In some embodiments, the second focus voltage ranges from 0.5 to 30, 0.5 to 20, 0.5 to 10, 0.5 to 9.5, 0.5 to 9, 0.5 to 8.5, 0.5 to 8, 0.5 to 7.5, 0.5 to 7, 0.5 to 6.5, 0.5 to 6, 0.5 to 5.5, 0.5 to 5, 0.5 to 4.5, 0.5 to 4, 0.5 to 3.5, 0.5 to 3, 0.5 to 2.5, 0.5 to 2, 0.5 to 1.5, 0.5 to 1, 0.5 to 0.9, 0.5 to 0.8, 0.5 to 0.7, 0.5 to 0.6, 0.7 to 30, 0.7 to 20, 0.7 to 10, 0.7 to 9.5, 0.7 to 9, 0.7 to 8.5, 0.7 to 8, 0.7 to 7.5, 0.7 to 7, 0.7 to 6.5, 0.7 to 6, 0.7 to 5.5, 0.7 to 5, 0.7 to 4.5, 0.7 to 4, 0.7 to 3.5, 0.7 to 3, 0.7 to 2.5, 0.7 to 2, 0.7 to 1.5, 0.7 to 1, 1 to 30, 1 to 20, 1 to 10, 1 to 9.5, 1 to 9, 1 to 8.5, 1 to 8, 1 to 7.5, 1 to 7, 1 to 6.5, 1 to 6, 1 to 5.5, 1 to 5, 1 to 4.5, 1 to 4, 1 to 3.5, 1 to 3, 1 to 2.5, 1 to 2, 1 to 1.5, 2 to 30, 2 to 20, 2 to 10, 2 to 9.5, 2 to 9, 2 to 8.5, 2 to 8, 2 to 7.5, 2 to 7, 2 to 6.5, 2 to 6, 2 to 5.5, 2 to 5, 2 to 4.5, 2 to 4, 2 to 3.5, 2 to 3, or 2 to 2.5, 3 to 10, 3 to 9.5, 3 to 9, 3 to 8.5, 3 to 8, 3 to 7.5, 3 to 7, 3 to 6.5, 3 to 6, 3 to 5.5, 3 to 5, 3 to 4.5, 3 to 4, or 3 to 3.5 kV.

Compound specific voltage setting described herein may result in simultaneous detection of N-nitrosamines and NDSRI compounds with optimum sensitivity avoiding separate testing of same sample.

In some embodiments, the second ionizing comprises applying a second voltage to the sample. In some embodiments, the second ionizing comprises applying a second voltage to at least one compound of the plurality of compounds that is readily ionizable under APCI conditions. In some embodiments, the second ionizing comprises applying a second voltage to at least one nitrosamine in the sample. In some embodiments, the second ionizing comprises applying a second voltage to all compounds in the sample that are readily ionizable under APCI conditions, irrespective of their elution. In some embodiments, the second ionizing comprises applying a second voltage to all N-nitrosamines in the sample.

Herein “readily ionizable” may include compound that is ionized immediately under ESI condition or APCI conditions. Being “under ESI condition” may mean when the ESI method described herein is applied to a sample containing the compound. Being “under APCI condition” may mean when the APCI method described herein is applied to a sample containing the compound.

In some embodiments, the method described herein is performed in one device. The device may comprise multiple parts that are physically connected. The device may comprise an ESI probe. The device may comprise a liquid chromatograph mass spectrometer described herein. In some embodiments, the sample is injected into the device once in performing the method described herein. In some embodiments, the first and second ionized compounds are detected simultaneously.

Hereinafter, the present disclosure will be described in more detail with reference to the following examples. But the following Examples are intended to illustrate the present embodiments, and the scope of the Examples is not limited thereto only.

### EXAMPLES

Conventional ESI or APCI LC-MS/MS methods for Nitrosamines and NDSRI respectively require multiple testing for single product (i.e. 2 testing methods for one each for Nitrosamine and NDSRI using APCI and ESI respectively. This significantly affects time, resources, chemical consumption, and overall throughput. Selecting any single technique (i.e. either ESI or APCI) for simultaneous detection of both Nitrosamines and NDSRI results in compromised sensitivities e.g. Nitrosamines having poor response in ESI whereas NDSRI shows poor sensitivity in APCI.

This example caters to the need of having a unified LC-MS/MS method for simultaneous detection of both these types of compounds which is challenging due to poor sensitivity of Nitrosamines on ESI and major NDSRIs on APCI. A novel, hybrid LC-MS/MS source available in Shimadzu's triple quadrupole LCMS systems called as the dual ionization source (DUIS) is equipped with capabilities of an ESI and APCI within same source. This allows a simultaneous detection of both Nitrosamines and NDSRIs by selectively altering the source parameters where compound dependent ESI-like or APCI-like conditions can be set and desired sensitivities can be achieved for both. The ESI predominant conditions can be achieved by setting an optimized interface voltage applied to the discharge electrodes installed in the electrospray ionization unit and atmospheric pressure chemical ionization unit.

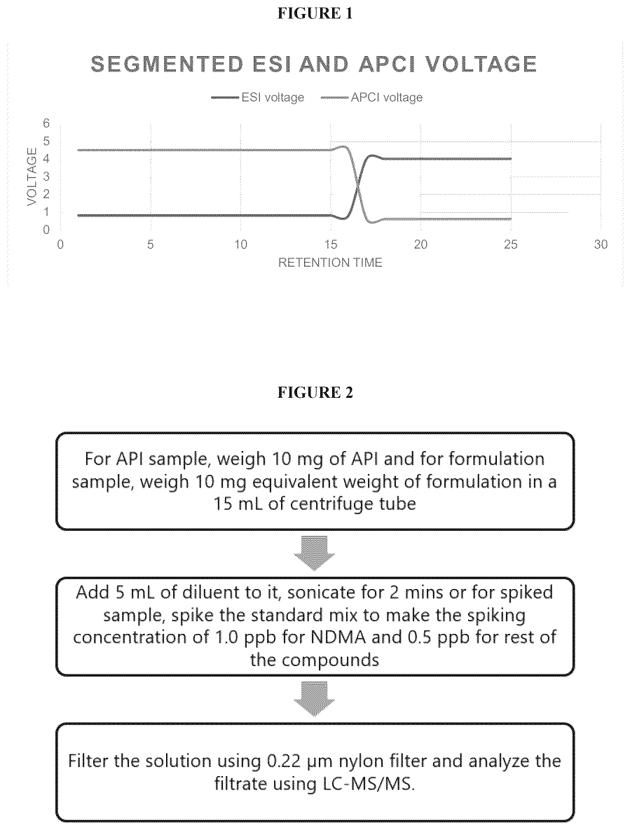

FIG. 1 shows a graphical representation of ESI and APCI voltage change programed which is only for demonstration purposes.

The effectiveness of this example may come from the flexibility of setting compound dependent ESI-like or APCI-like voltages provided by DUIS ionization source. This may give a simultaneous LC-MS/MS method for low level detection of both Nitrosamines and NDSRI. This approach may deliver an uncompromised sensitivity for all the compounds along with cost saving, speed & throughput.

N-nitrosamines may be monitored using atmospheric pressure chemical ionization (APCI) in LCMS analysis. This preference is due to their smaller molecular size, which can result in higher background interference when using electrospray ionization (ESI). In contrast, NDSRIs may be analyzed using ESI because they exhibit higher sensitivity with this ionization method. Considering the above scenario, API and formulations with potential of having presence of both N-nitrosamines and NDSRIs may be tested with multiple methods. Consumption of chemicals, reagents and decreasing throughput are some of the drawbacks associating with such multiple testing. This leads to a high demand for having a method with simultaneous detection of both N-nitrosamines and NDSRI using LC-MS/MS. For simultaneous detection, a dual ionization source (DUIS) can be useful as it can ionize both the N-nitrosamines and NDSRIs using same interface. Optimum sensitivities for individual compounds can be achieved by optimizing the source conditions to either have ESI-like or APCI-like conditions. This example describes a LC-MS/MS procedure for quantitation of nine N-nitrosamines and NDSRI in Afatinib API and formulation which was performed using an Ultra High Performance Liquid Chromatograph (UHPLC) Nexera™ X3 coupled with LCMS-8060NX, a Triple Quadrupole Mass Spectrometer equipped with a DUIS source from Shimadzu Corporation, Japan.

The nine N-nitrosamines and one NDSRI includes N-nitroso-dimethylamine (NDMA), N-nitrosomethylethylamine (NMEA), N-Nitrosopyrrolidine (NPYR), N-nitroso-diethylamine (NDEA), N-nitrosopiperidine (NPIP), N-nitroso-ethyl-isopropylamine (NEIPA), N-nitroso-diisopropylamine (NDIPA), N-nitroso-dipropylamine (NDPA), N-nitroso-dibutylamine (NDBA) and N-nitroso Afatinib impurity-2 (N-AFA).

Individual standards for all nine N-nitrosamines and NDSRI were purchased locally. Stock solutions for individual N-nitrosamines and NDSRI were prepared and analysed in scan mode. Further, steps such as precursor ion selection, Multiple Reaction Monitoring (MRM) optimization at different Collision Energies (CE) and voltage optimization were performed using Shimadzu's LabSolutions™ auto MRM optimization feature to obtain MRMs and their optimum CEs. To achieve optimum sensitivity, parameters such as interface voltage and focus voltage were fine tunned. An LC method (Table 1) was developed with an aim to separate 10 compounds and API under study which was achieved using Shimadzu make Shim-pack Scepter™ PFPP (Metal free), 2.1 mm I.D.×100 mm and 3.0 μm LC column (P/N: 227-31089-02).

TABLE 1
Instrument parameters for LC-MS/MS

| | |
|---|---|
| HPLC System | Nexera X3 |
| Column | Shim-pack Scepter PFPP-120 (Metal free) 2.1 mm |
| | I.D. × 100 mm, 3 μm (P/N: 227-31089-02) |
| Column Temp. | 45° C. |
| Mobile Phases | A: 10 mM ammonium formate in water |
| | B: Methanol:Acetonitrile (9:1) v/v |
| Flow Rate | 0.45 mL/min |
| Gradient Program | B Conc. 2% (0-3 min)→40% (8 min)→70% (20 |
| (B %) | min)→100% (21-25 min)→ 2% (25.5-35 min) |
| Injection Volume | 40 μL |
| MS | LCMS-8060NX |
| Ionization Source | DUIS ™ |
| Temperature | Interface: 400° C. |
| | Desolvation Line: 250° C. |
| | Heater Block: 400° C. |
| Gas Flows | Nebulizing Gas: 3.0 L/min |
| | Drying Gas: 4.0 L/min |

Optimized MRMs for individual compounds are listed in Table 2. For quantitation, a linearity ranging from 1.0-20.0 ppb for NDMA, 0.5-20.0 ppb for NMEA, NPYR, NPIP, NDEA, NEIPA, NDIPA, NDPA, NDBA and 0.5-7.5 ppb for N-AFA was prepared in diluent and was analyzed using LC-MS/MS. The limit of quantitation (LOQ) was found to be 1.0 ppb for NDMA and 0.5 ppb for rest of the compounds. The S/N and % RSD at LOQ are shown in Table 3.

TABLE 2
MRM transitions for 9 N-nitrosamines and 1 NDSRI

| Compound | Precursor m/z | Product m/z | CE |
|---|---|---|---|
| NDMA | 74.90 | 58.05 | −10 |
| NMEA | 89.20 | 61.25 | −15 |
| NPYR | 101.00 | 55.25 | −18 |
| NDEA | 103.00 | 29.05 | −16 |
| NPIP | 115.05 | 41.00 | −22 |
| NEIPA | 117.00 | 75.10 | −13 |
| NDIPA | 131.10 | 89.15 | −12 |
| NDPA | 131.00 | 89.05 | −12 |
| NDBA | 159.00 | 41.05 | −22 |
| N-AFA | 501.05 | 414.00 | −23 |

TABLE 3
Coefficient of determination tor calibration curves,
repeatability of area for LOQ solution and S/N ratio
for LOQ solution (Conc. expressed are as such)

| | | CC | LOQ | | |
|---|---|---|---|---|---|
| | | Range | Conc. | % RSD | |
| Abbr. | r2 | (ppb) | (ppb) | (n = 6) | S/N |
| NDMA | 0.993 | 1.0-20.0 | 1.0 | 11.0 | 30 |
| NMEA | 0.999 | 0.5-20.0 | 0.5 | 8.2 | 52 |
| NPYR | 0.998 | 0.5-20.0 | 0.5 | 12.3 | 66 |
| NDEA | 0.999 | 0.5-20.0 | 0.5 | 11.0 | 89 |
| NPIP | 0.999 | 0.5-20.0 | 0.5 | 7.4 | 77 |
| NEIPA | 0.999 | 0.5-20.0 | 0.5 | 4.9 | 198 |
| NDIPA | 0.999 | 0.5-20.0 | 0.5 | 11.3 | 101 |
| NDPA | 0.999 | 0.5-20.0 | 0.5 | 8.5 | 86 |
| NDBA | 0.999 | 0.5-20.0 | 0.5 | 4.0 | 142 |
| N-AFA | 0.994 | 0.5-7.5 | 0.5 | 8.4 | 609 |

Abbr. = Abbreviation;
CC = Calibration curve;
Conc. = Concentration;
S/N = Signal-to-noise ratio

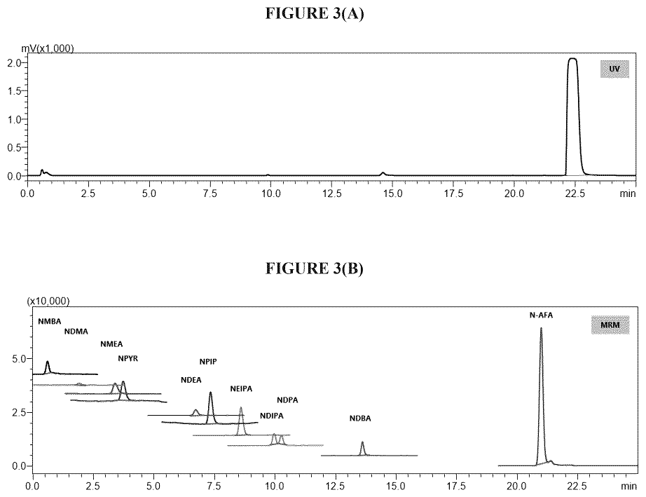

FIG. 2 depicts the sample preparation protocol for API and formulation samples and spiked samples. Briefly, 10 mg of API sample and 10 mg equivalent weight of formulation were placed in a 10 mL centrifuge tube. Then, add 5 mL of diluent into the tube, sonicate for 2 minutes for spiked sample. Spike the standard mix to make the concentration of 1.0 ppb for NDMA and 0.5 ppb for rest of the compounds. Then, filter the solution using 0.22 μm nylone filter and analyze the filtrate using LC-MS/MS.

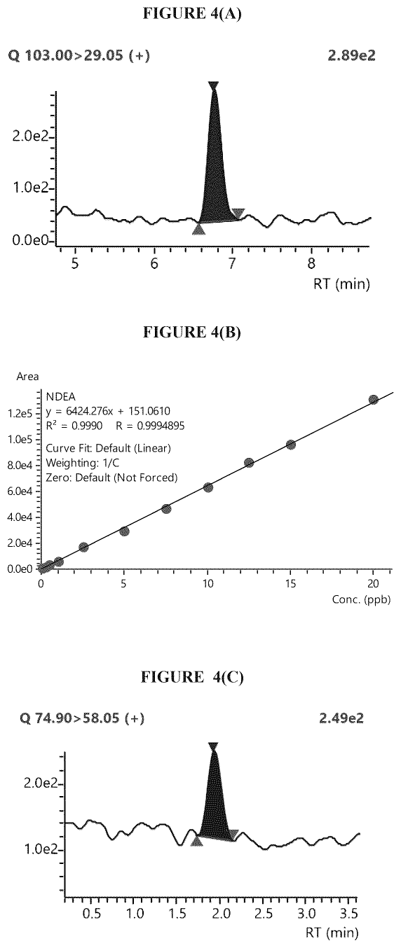

Results of analysis are shown in FIGS. 3(A) and 3(B). The chromatographic peak separation between Afatinib API detected in UV are shown in FIG. 3(A) and Nitrosamines and N-AFA detected in MRM mode are shown in FIG. 3(B). The results shows that well-balanced MS chromatograms were obtained.

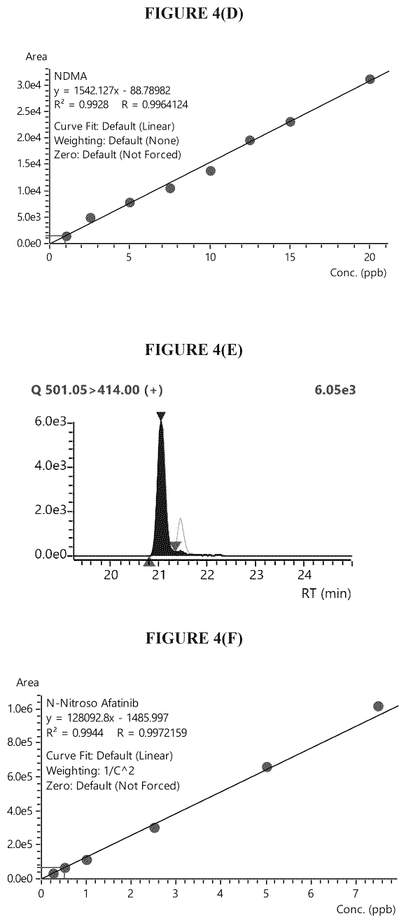

Chromatogram for LOQ level solution for NDEA, NDMA, and N-AFA were shown in FIGS. 4(A), 4(C), and 4(E) and calibration curve were shown in FIGS. 4(B), 4(D), and 4(F). The summary of content present in API and formulation samples as well as the recovery summary performed in API sample is summarized in Table 4 and 5, respectively.

TABLE 4
Summary of concentrations obtained in Afatinib
API, and formulation samples are shown below
Content in Afatinib sample (ppb)

| Abbr. | API | Formulation |
|---|---|---|
| NDMA | Below LOQ | Below LOQ |
| NMEA | Below LOQ | Below LOQ |
| NPYR | Below LOQ | Below LOQ |
| NDEA | Below LOQ | Below LOQ |
| NPIP | Below LOQ | Below LOQ |
| NEIPA | Below LOQ | Below LOQ |
| NDIPA | Below LOQ | Below LOQ |
| NDPA | Below LOQ | Below LOQ |
| NDBA | Below LOQ | Below LOQ |
| N-AFA | Below LOQ | Below LOQ |

TABLE 5
Summary for samples spiked at 500 ppb for NDMA and 250 ppb for rest
of the compounds (Results expressed are relative to sample concentration)
% Recoveries of N-nitrosamines and Nitroso Afatinib in Afatinib API

| | Amt. in | Amt. | Amt. | |
|---|---|---|---|---|
| | sample | obtained | spiked | % |
| Abbr. | (ppb) | (ppb) | (ppb) | Rec. |
| NDMA | Below LOQ | 433.7 | 500 | 87 |
| NMEA | Below LOQ | 252.8 | 250 | 101 |
| NPYR | Below LOQ | 304.4 | 250 | 122 |
| NDEA | Below LOQ | 292.1 | 250 | 117 |
| NPIP | Below LOQ | 252.8 | 250 | 101 |
| NEIPA | Below LOQ | 251.7 | 250 | 101 |
| NDIPA | Below LOQ | 250.0 | 250 | 100 |
| NDPA | Below LOQ | 294.1 | 250 | 118 |
| NDBA | Below LOQ | 253.3 | 250 | 101 |
| N-AFA | Below LOQ | 219.6 | 250 | 88 |

% Rec. = Percentage recovery;
Amt. = Amount

Table 6 shows the voltage summary set for individual compounds.

TABLE 6
NSA-NDSRI using DUIS Voltage Summary

| | Interface | Focus |
|---|---|---|
| | Voltage | Voltages |
| Compound | (kV) | (kV) |
| NMBA | 0.8 | 4.0 |
| NDMA | 0.8 | 4.5 |
| NMEA | 0.8 | 4.0 |
| NPYR | 0.8 | 4.0 |
| NDEA | 0.8 | 4.0 |
| NPIP | 0.8 | 4.0 |
| NEIPA | 0.8 | 4.0 |
| NDIPA | 0.8 | 4.0 |
| NDPA | 0.8 | 4.0 |
| NDBA | 0.8 | 4.0 |
| N-Nitroso Afatinib imp | 1.0 | 2.0 |

Quantitation of 9 N-nitrosamines and NDSRI in Afatinib API and formulation was successfully demonstrated on Shimadzu LCMS-8060NX equipped with DUIS source. Repeatability for N-nitrosamines and NDSRI was found to be less than 15.0%. Recoveries for all N-nitrosamines and NDSRI were found to be between 85-125%. The newly developed IonFocus™ ion source unit and patented lens system (UF-Qarray™ II) of LCMS-8060NX improves system robustness by efficiently introducing only ions into the mass spectrometer and removing unwanted neutral particles and contaminants. Revolutionary DUIS ion source with ion focus technology not only detect compounds of diverse nature but also improves baseline by eliminating contaminants.

We claim:

1. A method of at least one of a detecting nitrosamine drug substance related impurities (NDSRI) and at least one of a nitrosamine compound from an active pharmaceutical ingredient (API), the method comprising;
first ionizing the NDSRI with a first ionization method to produce ionized NDSRI,
second ionizing the nitrosamine compound with a second ionization method to produce ionized nitrosamine compound, and
detecting the ionized NDSRI and the ionized nitrosamine compound, wherein the first ionization method and the second ionization method are different.

2. The method according to claim 1, wherein the first and second ionizing steps are performed alternately.

3. The method according to claim 1, wherein the first ionization method is Electrospray Ionization (ESI) method and the second ionization method is Atmospheric Pressure Chemical Ionization (APCI) method.

4. The method according to claim 1, wherein the nitrosamine compound comprises an N-nitrosamine compound.

5. The method according to claim 4, wherein the N-nitrosamine compound comprises at least one selected from the group consisting of N-nitroso-dimethylamine (NDMA), N-nitrosomethylethylamine (NMEA), N-Nitrosopyrrolidine (NPYR), N-nitroso-diethylamine (NDEA), N-nitrosopiperidine (NPIP), N-nitroso-ethyl-isopropylamine (NEIPA), N-nitroso-diisopropylamine (NDIPA), N-nitroso-dipropylamine (NDPA), and N-nitroso-dibutylamine (NDBA).

6. The method according to claim 1, wherein the first ionizing comprises selecting a interface voltage selected from 0.1 kV to 2.0 kV.

7. The method according to claim 1, wherein the first ionizing comprises selecting a first focus voltage selected from 1.0 kV to 3.0 kV.

8. The method according to claim 1, wherein the second ionizing comprises selecting a second interface voltage selected from 0.5 kV to 1.5 kV.

9. The method according to claim 1, wherein the second ionizing comprises selecting a focus voltage selected from 3.0 kV to 5.0 kV.

10. The method according to claim 1, wherein the ionized NDSRI and the ionized nitrosamine compound are detected simultaneously.
